(19) United States
(12) Patent Application Publication
(10) Pub. No.: US 2005/0202492 A1
(43) Pub. Date: Sep. 15, 2005

(54) Microarray having probe polynucleotide spots capable of binding to the same target polynucleotide fragment maximally separated from each other and a method of producing the same

(76) Inventors: Yun-sun Nam, Seoul (KR); Nam Huh, Seoul (KR); Hyeong-sop Shim, Hwaseong-si (KR); Ji-young Oh, Yongin-si (KR)

Correspondence Address: CANTOR COLBURN, LLP, 55 GRIFFIN ROAD SOUTH, BLOOMFIELD, CT, 06002, US

(21) Appl. No.: 11/078,601

(22) Filed: Mar. 11, 2005

(30) Foreign Application Priority Data
Mar. 12, 2004 (KR) 10-2004-0017026

(51) Int. Cl. C12Q 1/68; C12M 1/34; A61L 2/00

(52) U.S. Cl. 435/6; 435/287.2; 427/2.11

(57) ABSTRACT

The present invention relates to a microarray having probe polynucleotide spots capable of binding to the same target polynucleotide fragment maximally separated from each other, and a method of producing the same.

### BACKGROUND OF THE INVENTION

This application claims the benefit of Korean Patent Application No. 10-2004-0017026, filed on Mar. 12, 2004, in the Korean Intellectual Property Office, the disclosure of which is incorporated herein in its entirety by reference.

1. Field of the Invention

The present invention relates to a microarray having probe polynucleotide spots capable of binding to the same target polynucleotide fragment maximally separated from each other, and a method of producing the same.

2. Description of the Related Art

“Polynucleotide microarrays” or “nucleic acid microarrays” refer to substrates having polynucleotides immobilized on predetermined regions at a high density. Such microarrays are well known in the art, and examples are disclosed in U.S. Pat. Nos. 5,445,934 and 5,744,305. Generally, microarrays are produced by using photolithographic technology or by spotting. In the photolithographic method, a predetermined region of a substrate coated with a monomer having a removable protecting group is exposed to an energy source to remove the protecting group from the monomer, and then a second monomer having a removable protecting group is coupled to the monomer. The process of exposure to an energy source, removal of the protecting group, and coupling of a monomer is repeated to produce a desired polynucleotide on the substrate. In the spotting method, a previously synthesized polynucleotide is immobilized at a predetermined location on a substrate. Polynucleotide microarrays and methods of producing the same are described in U.S. Pat. Nos. 5,744,305, 5,143,854 and 5,424,186, the disclosures of which are incorporated herein in their entirety by reference.

In the conventional polynucleotide microarrays, a polynucleotide immobilized on a predetermined region of the substrate is referred to as “probe polynucleotide”. Generally, a probe polynucleotide has a sequence complementary to a sequence of a target polynucleotide, thus capable of sequence-specifically binding to the target polynucleotide in a hydridization process. The sequence-specific binding between the probe polynucleotide and the target polynucleotide can be used to detect the target polynucleotide. Generally, the term “spot” means a predetermined location of a substrate on which polynucleotide groups are immobilized. In a microarray, spots are arranged at a high density, for example, at least 10,000 spots/cm², on a substrate.

In a conventional microarray, spots are randomly arranged, irrespective of polynucleotide sequence. If a target nucleic acid molecule binds to two or more probe polynucleotides, this can have an adverse effect on the analytical results of the target nucleic acid molecule using the microarray. That is, if a target molecule binds to at least two probe polynucleotides in a binding region, at least two probe polynucleotides compete with each other for one target molecule. Thus, there is a need for higher concentration of target molecules in a local region. However, a diffusion rate of a target nucleic acid is limited, and thus increasing a concentration of the target nucleic acid in a specific region is limited. Diffusion coefficient of a DNA fragment in water, Dw, may be represented as 4.9×10⁻⁶ cm²/s×[bp]^{-0.72}. As the size of a DNA fragment becomes greater, the diffusion coefficient becomes smaller (“J. Bio. Chem.”, vol. 275 (2000), pp. 1625-1629). Thus, a diffusion coefficient is limited. Thus, the analytical results of the target nucleic acid when using a polynucleotide microarray produced using a conventional method of arranging spots may be different according to the microarray to be tested even under the same experimental conditions.

Thus, there is a need for a method of arranging spots on a polynucleotide microarray so that the concentration of target nucleic acid to be hybridized to a probe polynucleotide may not be limited. The present inventors conducted research to develop a method of arranging spots on a polynucleotide so that probe polynucleotide spots capable of binding to the same target nucleic acid molecule may be maximally separated from each other.

### SUMMARY OF THE INVENTION

The present invention provides a high quality polynucleotide microarray capable of providing good analytical results of a target nucleic acid.

The present invention also provides a method of producing the above polynucleotide microarray.

According to an aspect of the present invention, there is provided a polynucleotide microarray having at least two probe polynucleotides capable of binding to the same target polynucleotide immobilized thereon, wherein a spot region on a substrate of the polynucleotide microarray is segmented into blocks arranged in rows and columns adjacent to each other, the number of blocks being 2 m, wherein m is the maximum value of the numbers of the probe polynucleotides capable of binding to the same target polynucleotide, and the spots are arranged such that each block has a width-length ratio of \sqrt{3}:1, the blocks are designated as one of block of type a and block of type b so that two blocks of the same type, a or b, do not have sides adjacent to each other, and the probe polynucleotides are immobilized on the block of type a or block of type b so that probe polynucleotides capable of binding to the same target polynucleotide are immobilized at the same corresponding location in selected block of type a or block of type b.

According to another aspect of the present invention, there is provided a method of producing a polynucleotide microarray having at least two probe polynucleotides capable of binding to the same target polynucleotide immobilized as spots thereon, comprising: segmenting a spot region of a substrate of the polynucleotide microarray into blocks arranged in rows and columns adjacent to each other, the number of blocks being 2 m, wherein m is the maximum value of the numbers of the probe polynucleotides capable of binding to the same target polynucleotide, and arranging the spots such that each block has a width-length ratio of \sqrt{3}:1, designating the blocks as one of block of type a and block of type b so that two blocks of the same type, a or b, do not have sides adjacent to each other, and immobilizing the probe polynucleotides on block of type a or block of type b so that probe polynucleotides capable of binding to the same target polynucleotide are immobilized at the same corresponding location in selected block of type a or block of type b.

### BRIEF DESCRIPTION OF THE DRAWINGS

The above and other features and advantages of the present invention will become more apparent by describing in detail exemplary embodiments thereof with reference to the attached drawing in which:

FIG. 1 is a graph illustrating coefficients of variance (CV) for fluorescence intensity when using a polynucleotide microarray according to an embodiment of the present invention and when using a control microarry.

### DETAILED DESCRIPTION OF THE INVENTION

According to an embodiment of the present invention, there is provided a polynucleotide microarray having at least two probe polynucleotides capable of binding to the same target polynucleotide immobilized thereon, wherein a spot region on a substrate of the polynucleotide microarray is segmented into blocks arranged in rows and columns adjacent to each other, the number of blocks being 2m, wherein m is the maximum value of the numbers of the probe polynucleotides capable of binding to the same target polynucleotide, and the spots are arranged such that each block has a width-length ratio of \sqrt{3}:1, the blocks are designated as one of block of type a and block of type b so that two blocks of the same type, a or b, do not have sides adjacent to each other, and the probe polynucleotides are immobilized on the block of type a or block of type b so that probe polynucleotides capable of binding to the same target polynucleotide are immobilized at the same corresponding location in selected block of type a or block of type b.

In the polynucleotide microarray, rows or columns of the blocks may be separated.

The polynucleotide microarray according to the embodiment of the present invention has the probe polynucleotide spots capable of binding to the same target polynucleotide maximally separated from each other. Thus, by using the polynucleotide microarray, it is possible to reduce variations in analytical tests for the target nucleic acid.

In the polynucleotide microarray according to the present embodiment, a spot region on a substrate is segmented into blocks arranged in rows and columns adjacent to each other, the number of blocks being 2 m, wherein m is the maximum value of the numbers of the probe polynucleotides capable of binding to the same target polynucleotide. It is not necessary that the blocks be adjacent to each other. A space may be provided between rows or between columns of the blocks to reduce interference of the fluorescence intensity in measuring the hybridization between the probe polynucleotides and the target polynucleotide. For example, if the number of probe polynucleotides capable of binding to target nucleic acid T1 is 2 and the number of probe polynucleotides capable of binding to target nucleic acid T2 is 5, then m is 5 and the number of blocks in the microarray is 10. In addition, the spots are arranged so that each block has a width-length ratio of \sqrt{3}:1. The expression “each block has a width-length ratio of \sqrt{3}:1” means that a ratio of the number of probe polynucleotides immobilized along a width of each block to the number of probe polynucleotides immobilized along a length of each block is an integer ratio approximating about 1.7:1, for example, 2:1 or 3:2.

In the polynucleotide microarray according to an embodiment of the present invention, the blocks are designated as one of two types so that two blocks of the same type do not have sides adjacent to each other. When the number of columns of blocks is 4 and m is 8, the designation to one of the two types of blocks is performed as follows. A total of 16 blocks (2 m=16) are arranged in 4 rows and 4 columns, and then the blocks are designated as one of the two types of blocks, a and b, so that two block of type a or two block of type b do not have sides adjacent to each other, as illustrated in Table 1.

TABLE 1
Designations to one of blocks a and b when the
number of columns of blocks is an even number

| | | | |
|---|---|---|---|
| a | b | a | b |
| b | a | b | a |
| a | b | a | b |
| b | a | b | a |

When the number of rows of blocks is an odd number, the designation may be performed in the same manner as when the number of blocks is even. For example, when the number of columns of blocks is 5 and m is 10, the designation to one of the two types of blocks is performed as follows. A total of 20 blocks (2 m=20) are arranged in 4 rows and 5 columns, and then the blocks are designated as one of the two types of blocks a and b so that two block of type a or two block of type b do not have sides adjacent to each other, as illustrated in Table 2.

TABLE 2
Designations to one of blocks a and b when the
number of columns of blocks is an odd number

| | | | | |
|---|---|---|---|---|
| a | b | a | b | a |
| b | a | b | a | b |
| a | b | a | b | a |
| b | a | b | a | b |

After the designations to one of the two types of blocks a and b in the polynucleotide microarray, the probe polynucleotides are immobilized on block a or b so that each probe polynucleotide capable of binding to the same target polynucleotide is immobilized at the same corresponding location in selected block of type a or block of type b.

According to another embodiment of the present invention, there is provided a method of producing a polynucleotide microarray having at least two probe polynucleotides capable of binding to the same target polynucleotide immobilized as spots thereon, comprising: segmenting a spot region of a substrate of the polynucleotide microarray into blocks arranged in rows and columns adjacent to each other, the number of blocks being 2 m, wherein m is the maximum value of the numbers of the probe polynucleotides capable of binding to the same target polynucleotide, and arranging the spots such that each block has a width-length ratio of \sqrt{3}:1, designating the blocks as one of block of type a and block of type b so that two blocks of the same type, a or b, do not have sides adjacent to each other, and immobilizing the probe polynucleotides on block of type a or block of type b so that probe polynucleotides capable of binding to the same target polynucleotide are immobilized at the same corresponding location in selected block of type a or block of type b.

The method of producing a polynucleotide microarray may further comprise providing a space between the blocks.

According to the method of producing a polynucleotide microarray, at least two probe polynucleotides capable of binding to the same target polynucleotide can be maximally separated from each other in the polynucleotide microarray.

First, a spot region on the substrate is segmented into blocks arranged in rows and columns adjacent to each other, the number of blocks being 2 m, wherein m is the maximum value of the numbers of probe polynucleotides capable of binding to the same target polynucleotide. Each block is formed with a width-length ratio of \sqrt{3}:1 and the probe polynucleotide spots are arranged to produce a ratio of about 1.7:1. That is, a ratio of the number of probe polynucleotides immobilized in a width of each block to the number of probe polynucleotides immobilized in a length of each block is an integer ratio approximating about 1.7:1, for example, 2:1, 3:2 etc.

Next, the blocks are designated as one of the two types of blocks a and b so that two of the same type of blocks, i.e., a or b, do not have sides adjacent to each other. Then, the probe polynucleotides are immobilized on block of type a or b so that probe polynucleotides capable of binding to the same target polynucleotide are immobilized at the same corresponding location in a selected block of type a or block of type b.

The designations to one of the two types of blocks may be performed in the same manner as explained for the polynucleotide microarray according to an embodiment of the present invention.

The present method of producing a polynucleotide microarray can be applied to the conventional method for immobilizing a probe polynucleotide on a microarray, for example, a photolithographic method or a spotting method. Polynucleotide microarrays and methods of producing the same are described in U.S. Pat. Nos. 5,744,305, 5,143,854 and 5,424,186, the disclosures of which are incorporated herein in their entirety by reference. Examples to which the method of producing a microarray according to an embodiment of the present invention can be applied are not limited to the above photolithographic method and spotting method.

Hereinafter, the present invention will be described in more detail with reference to the following example. However, the example is provided for the purpose of illustration and is not intended to limit the scope of the invention.

### EXAMPLE

In this Example, using the method according to an embodiment of the present invention, at least two probe polynucleotides capable of binding to the same target nucleic acid were arranged on a substrate to produce a microarray. Then, hybridization with a target nucleic acid was performed on the microarray and the obtained data were analyzed. The experiment was performed as follows.

The number of target nucleic acids used in the experiment was 18 (SEQ ID Nos. 1 through 18) (see, Table 3). The number of types of probe polynucleotides which bind to the target nucleic acids was 78 (SEQ ID Nos. 19 through 96) (see, Table 4). The probe Pi,j binds to the target nucleic acid Ai. In addition to the 78 probes, control probes were arranged on the substrate and spaces of 4.5 mm were provided between rows and columns, crossing at the center of the substrate (referred to as “interval” in Tables 6 and 8). The maximum value m of the number of probe polynucleotides capable of binding to the same target nucleic acid was 11 (see, Table 5).

TABLE 3
Target nucleic acids

| Name | SEQ ID No. |
|---|---|
| A1 | 1 |
| A2 | 2 |
| A3 | 3 |
| A4 | 4 |
| A5 | 5 |
| A6 | 6 |
| A7 | 7 |
| A8 | 8 |
| A9 | 9 |
| A10 | 10 |
| A11 | 11 |
| A12 | 12 |
| A13 | 13 |
| A14 | 14 |
| A15 | 15 |
| A16 | 16 |
| A17 | 17 |
| A18 | 18 |

TABLE 4
Probe polynucleotides

| | SEQ ID |
|---|---|
| Probe | No. |
| P1,1 | 19 |
| P1,2 | 20 |
| P1,3 | 21 |
| P1,4 | 22 |
| P1,5 | 23 |
| P2,1 | 24 |
| P2,2 | 25 |
| P2,3 | 26 |
| P2,4 | 27 |
| P2,5 | 28 |
| P2,6 | 29 |
| P3,1 | 30 |
| P3,2 | 31 |
| P3,3 | 32 |
| P3,4 | 33 |
| P3,5 | 34 |
| P3,6 | 35 |
| P3,7 | 36 |
| P3,8 | 37 |
| P3,9 | 38 |
| P3,10 | 39 |
| P3,11 | 40 |
| P4,1 | 41 |
| P4,2 | 42 |
| P4,3 | 43 |
| P4,4 | 44 |
| P4,5 | 45 |
| P4,6 | 46 |
| P4,7 | 47 |
| P5,1 | 48 |
| P5,2 | 49 |
| P5,3 | 50 |
| P5,4 | 51 |
| P6,1 | 52 |
| P6,2 | 53 |
| P6,3 | 54 |
| P6,4 | 55 |
| P6,5 | 56 |
| P6,6 | 57 |
| P6,7 | 58 |
| P7,1 | 59 |
| P7,2 | 60 |
| P7,3 | 61 |
| P7,4 | 62 |
| P7,5 | 63 |
| P7,6 | 64 |
| P7,7 | 65 |
| P7,8 | 66 |
| P7,9 | 67 |
| P8,1 | 68 |
| P8,2 | 69 |
| P8,3 | 70 |
| P8,4 | 71 |
| P8,5 | 72 |
| P8,6 | 73 |
| P9,1 | 74 |
| P10,1 | 75 |
| P10,2 | 76 |
| P10,3 | 77 |
| P11,1 | 78 |
| P12,1 | 79 |
| P12,2 | 80 |
| P12,3 | 81 |
| P13,1 | 82 |
| P13,2 | 83 |
| P14,1 | 84 |
| P14,2 | 85 |
| P14,3 | 86 |
| P14,4 | 87 |
| P14,5 | 88 |
| P14,6 | 89 |
| P14,7 | 90 |
| P14,8 | 91 |
| P15,1 | 92 |
| P16,1 | 93 |
| P16,2 | 94 |
| P17,1 | 95 |
| P18,1 | 96 |

TABLE 5
Target nucleic acids and number of
probe polynucleotides capable of
binding to each target polynucleotide

| SEQ ID No. of | |
|---|---|
| target nucleic | Number of |
| acid | probes |
| 1 | 5 |
| 2 | 6 |
| 3 | 11 |
| 4 | 7 |
| 5 | 4 |
| 6 | 7 |
| 7 | 9 |
| 8 | 6 |
| 9 | 1 |
| 10 | 3 |
| 11 | 1 |
| 12 | 3 |
| 13 | 2 |
| 14 | 8 |
| 15 | 1 |
| 16 | 2 |
| 17 | 1 |
| 18 | 1 |

To ensure accuracy of the experiment, three spots were arranged in a row for each probe. The three spots were separated by 300 μm. Fluorescence intensity of the probe was obtained by measuring an average of fluorescence intensities for the three spots. Next, 24 blocks were arranged in rows and columns on the substrate, each block being composed of three or four rows and six columns of spots and classified into two types of blocks, as described above. The probes (SEQ ID Nos. 19 through 96) were respectively immobilized on one of the two types of blocks so that each probe polynucleotide capable of binding to the same target polynucleotide was immobilized at the same corresponding location in selected type of blocks. There blocks are illustrated in Table 6. For comparison, probe polynucleotides capable of binding to the same target polynucleotide were arranged adjacent to each other (see, Table 7). The experiments were respectively performed on thirty microarrays.

In Tables 6 and 7, each block composed of four rows and six columns of spots in the 1^{st} through 4^{th} rows, the 5^{th} through 8^{th} rows, the 13^{th} through 16^{th} rows, and the 17^{th} through 20^{th} rows among a total of 23 rows, while each block composed of three rows and six columns of spots in the 9^{th} through 11^{th} rows and the 21^{th} through 23^{th} rows. The term “Interval” means a space of 4.5 mm between the blocks. For each probe, three spots are arranged in a row. For example, in a cell of “P3,1 Control”, a total of six spots, i.e., three spots of P3,1 plus three control spots, are arranged in a row.

TABLE 6
Arrangement of probe polynucleotides according to the embodiment
of the present invention

| | | | | | | | | |
|---|---|---|---|---|---|---|---|---|
| P3,1 | Control | P7,1 | Control | Interval | P3,2 | Control | P7,2 | Control |
| P4,1 | Control | P8,1 | Control | Interval | P4,2 | Control | P8,2 | Control |
| P6,4 | Control | P1,1 | Control | Interval | P6,5 | Control | P1,2 | Control |
| P5,1 | Control | P14,1 | Control | Interval | P5,2 | Control | P16,2 | Control |
| P7,6 | Control | P3,5 | Control | Interval | P7,8 | Control | P3,7 | Control |
| P8,5 | Control | P4,5 | Control | Interval | P8,6 | Control | P4,6 | Control |
| P2,5 | Control | P12,1 | Control | Interval | P2,6 | Control | P12,2 | Control |
| P14,2 | Control | P13,1 | Control | Interval | P14,3 | Control | P13,2 | Control |
| P3,10 | Control | P16,1 | Control | Interval | P3,6 | Control | Control | Control |
| P6,2 | Control | P15,1 | Control | Interval | P7,4 | Control | Control | Control |
| Control | Control | P14,6 | Control | Interval | Control | Control | P14,7 | Control |
| Interval | Interval | Interval | Interval | Interval | Interval | Interval | Interval | Interval |
| P3,3 | Control | P7,3 | Control | Interval | P3,4 | Control | P7,5 | Control |
| P4,3 | Control | P8,3 | Control | Interval | P4,4 | Control | P8,4 | Control |
| P6,6 | Control | P1,3 | Control | Interval | P6,7 | Control | P1,5 | Control |
| P5,3 | Control | P2,3 | Control | Interval | P5,4 | Control | P2,4 | Control |
| P7,9 | Control | P3,8 | Control | Interval | P10,2 | Control | P3,9 | Control |
| P10,1 | Control | P4,7 | Control | Interval | P11,1 | Control | P6,1 | Control |
| P2,1 | Control | P12,3 | Control | Interval | P2,2 | Control | P9,1 | Control |
| P14,4 | Control | Control | Control | Interval | P14,5 | Control | Control | Control |
| P3,11 | Control | P17,1 | Control | Interval | P7,7 | Control | Control | Control |
| P6,3 | Control | P18,1 | Control | Interval | P1,4 | Control | Control | Control |
| Control | Control | P14,8 | Control | Interval | P10,3 | Control | Control | Control |

TABLE 7
Arrangement of probe polynucleotides according to the conventional
method

| | | | | | | | | |
|---|---|---|---|---|---|---|---|---|
| Control | Control | P2,1 | Control | Interval | P3,6 | Control | Control | Control |
| P1,1 | Control | P2,2 | Control | Interval | P3,7 | Control | P3,8 | Control |
| P1,2 | Control | P1,3 | Control | Interval | P3,9 | Control | P3,10 | Control |
| P2,3 | Control | P2,4 | Control | Interval | P4,5 | Control | P4,6 | Control |
| P2,5 | Control | P2,6 | Control | Interval | P4,7 | Control | P3,11 | Control |
| P1,4 | Control | Control | Control | Interval | P6,1 | Control | P6,2 | Control |
| P1,5 | Control | P3,1 | Control | Interval | P6,3 | Control | P5,1 | Control |
| P4,1 | Control | P4,2 | Control | Interval | P6,4 | Control | P6,5 | Control |
| P4,3 | Control | P3,2 | Control | Interval | P5,2 | Control | P5,3 | Control |
| P4,4 | Control | P3,3 | Control | Interval | P6,6 | Control | P5,4 | Control |
| P3,4 | Control | P3,5 | Control | Interval | P6,7 | Control | Control | Control |
| Interval | Interval | Interval | Interval | Interval | Interval | Interval | Interval | Interval |
| P8,1 | Control | P7,1 | Control | Interval | P10,3 | Control | Control | Control |
| P8,2 | Control | P7,2 | Control | Interval | P11,1 | Control | P12,1 | Control |
| P8,3 | Control | P8,4 | Control | Interval | P13,1 | Control | P12,2 | Control |
| P8,5 | Control | P7,3 | Control | Interval | P13,2 | Control | P12,3 | Control |
| P7,4 | Control | Control | Control | Interval | P15,1 | Control | P14,1 | Control |
| P7,5 | Control | P8,6 | Control | Interval | P14,2 | Control | P14,3 | Control |
| P7,6 | Control | Control | Control | Interval | P14,4 | Control | P14,5 | Control |
| P7,7 | Control | Control | Control | Interval | P14,6 | Control | P14,7 | Control |
| P7,8 | Control | P7,9 | Control | Interval | P14,8 | Control | P16,1 | Control |
| P9,1 | Control | P10,1 | Control | Interval | P16,2 | Control | P18,1 | Control |
| P10,2 | Control | Control | Control | Interval | P17,1 | Control | Control | Control |

Immobilization of the probe polynucleotide was performed as follows. An aminohexanoic acid group-modified oligomer was used as a 5′-end of each probe. Then, a homemade array was produced using a spotting solution composed of 2 μM of DNA, 6 mM of PEG 1000, and 250 μM of bicarbonate buffer (pH 9.0).

Each wild type of target nucleic acid labeled with Cy3-dUTP during the amplification of the target nucleic acid was hybridized to the probe polynucleotide immobilized on each microarray at 32° C. for 16 hours. Then, fluorescence intensity was measured using GenePix 4000B laser scanner (available from Axon) at a PMT voltage of 530 and a laser power of 100%.

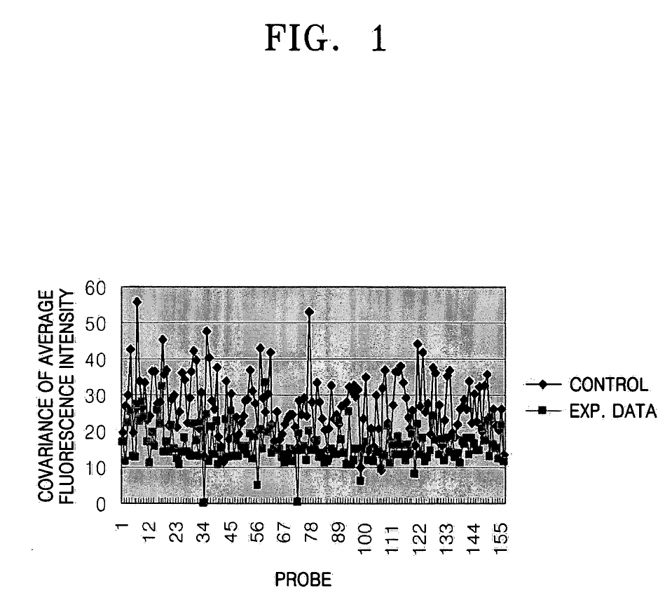

As a result, the microarray produced using the method according to an embodiment of the present invention has a remarkably lower coefficient of variance (CV) for fluorescence intensity than a control microarray. The determined CV values are graphed in FIG. 1. Thus, when a target nucleic acid is analyzed using the microarray produced using the method according to an embodiment of the present invention, it is possible to reduce variations in tests of the target nucleic acid, thus resulting in more objective experimental results.

The present invention provides a polynucleotide microarray permitting a reduction of variations in analytical tests of the target nucleic acid and a method of producing the same.

While the present invention has been particularly shown and described with reference to exemplary embodiments thereof, it will be understood by those of ordinary skill in the art that various changes in form and details may be made therein without departing from the spirit and scope of the present invention as defined by the following claims.

1. A polynucleotide microarray having at least two probe polynucleotides capable of binding to the same target polynucleotide immobilized thereon,
wherein a spot region on a substrate of the polynucleotide microarray is segmented into blocks arranged in rows and columns adjacent to each other, the number of blocks being 2 m, wherein m is the maximum value of the numbers of the probe polynucleotides capable of binding to the same target polynucleotide, and the spots are arranged such that each block has a width-length ratio of \sqrt{3}:1,
the blocks are designated as one of block of type a and block of type b so that two blocks of the same type, a or b, do not have sides adjacent to each other, and
the probe polynucleotides are immobilized on the block of type a or block of type b so that probe polynucleotides capable of binding to the same target polynucleotide are immobilized at the same corresponding location in selected block of type a or block of type b.

2. The polynucleotide microarray of claim 1, wherein the blocks are separated.

3. A method of producing a polynucleotide microarray having at least two probe polynucleotides capable of binding to the same target polynucleotide immobilized as spots thereon, comprising:
segmenting a spot region of a substrate of the polynucleotide microarray into blocks arranged in rows and columns adjacent to each other, the number of blocks being 2 m, wherein m is the maximum value of the numbers of the probe polynucleotides capable of binding to the same target polynucleotide, and arranging the spots such that each block has a width-length ratio of \sqrt{3}:1,
designating the blocks as one of block of type a and block of type b so that two blocks of the same type, a or b, do not have sides adjacent to each other, and
immobilizing the probe polynucleotides on block of type a or block of type b so that probe polynucleotides capable of binding to the same target polynucleotide are immobilized at the same corresponding location in selected block of type a or block of type b

4. The method of claim 2, further comprising forming a space between the blocks.


SEQUENCE LISTING (96 sequences)

SEQ ID NO: 1 - DNA, 419 bp, Artificial Sequence
  target polynucleotide A1
gggctcgttaggagctgaggggggcccctctaggctctcctgggagcggggacggggcagggctccttactgcagaagggtctccaccacggctttctggtgggccgcctcctcagggctgaggttctccagctctttgaggatgggtggcgtgaagtcttccccatcgtcgtccgtctcgtcctcggagccccgagtctcccccagcccattgggcagctcagccagctcccctcgaccgccgccgcaggactcccccttgtccagggggccttctccagccaggaggtagggccccggctcacccagtgcctggatcagtgcctctttgctcagccctgactcgagcagggccgccaggagctccgtctgcagctggctcagtttagaaaccatggctcggctgccacagggccacg

SEQ ID NO: 2 - DNA, 419 bp, Artificial Sequence
  target polynucleotide A2
cgtggccctgtggcagccgagccatggtttctaaactgagccagctgcagacggagctcctggcggccctgctcgagtcagggctgagcaaagaggcactgatccaggcactgggtgagccggggccctacctcctggctggagaaggccccctggacaagggggagtcctgcggcggcggtcgaggggagctggctgagctgcccaatgggctgggggagactcggggctccgaggacgagacggacgacgatggggaagacttcacgccacccatcctcaaagagctggagaacctcagccctgaggaggcggcccaccagaaagccgtggtggagacccttctgcagtaaggagccctgccccgtccccgctcccaggagagcctagaggggcccccctcagctcctaacgagccc

SEQ ID NO: 3 - DNA, 326 bp, Artificial Sequence
  target polynucleotide A3
gggatggtgaagcttccagcccccacctatgagttaggggagagtcctgggccctcccagggaagatgcggggtagggtcattacttacgctgcgccacctctcgctgcttgcggacgtaccaggtgtacagggcggcccgcttctgcgtcttcatgggagtgcccttgttgaggtgttgggacaggtgggactggttgaggccagtggtatcgaccacctcccgctgtgggatgttgtgctgctgcaggtaggacttgaccatcttcgccacacgccacgggtcctccctgggagagggcaaggacgggatctgctcagcaaggg

SEQ ID NO: 4 - DNA, 326 bp, Artificial Sequence
  target polynucleotide A4
cccttgctgagcagatcccgtccttgccctctcccagggaggacccgtggcgtgtggcgaagatggtcaagtcctacctgcagcagcacaacatcccacagcgggaggtggtcgataccactggcctcaaccagtcccacctgtcccaacacctcaacaagggcactcccatgaagacgcagaagcgggccgccctgtacacctggtacgtccgcaagcagcgagaggtggcgcagcgtaagtaatgaccctaccccgcatcttccctgggagggcccaggactctcccctaactcataggtgggggctggaagcttcaccatccc

SEQ ID NO: 5 - DNA, 270 bp, Artificial Sequence
  target polynuceotide A5
cgccgttgtacctattgcactcctccactagcgtctctcgctcctccttgctagggttcttctgcctctcataggcctggaacaggatctgctgggatgctgggccccacttgaaacggttcctccgccccttcttggttggtagctcatcacctgtgggctcttcaatcagccctccctgccctgcatgggtgaactctgcaggcacagaaagccgtgagtggggtactggccactctcaccttcccacgtccattcccctgaccttgc

SEQ ID NO: 6 - DNA, 270 bp, Artificial Sequence
  target polynucleotide A6
gcaaggtcaggggaatggacgtgggaaggtgagagtggccagtaccccactcacggctttctgtgcctgcagagttcacccatgcagggcagggagggctgattgaagagcccacaggtgatgagctaccaaccaagaaggggcggaggaaccgtttcaagtggggcccagcatcccagcagatcctgttccaggcctatgagaggcagaagaaccctagcaaggaggagcgagagacgctagtggaggagtgcaataggtacaacggcg

SEQ ID NO: 7 - DNA, 296 bp, Artificial Sequence
  target polynucleotide A7
cgtgtcccttgtccccacataccacttaccgtggaccttactgggggagagggcaggtggaggcaggccaggggagctgtgagcgggcagcgcaggtcccgggcctggccctggggggggcccgctgtacgtgtccatggccagcttgtgccggaaggcttcttctttgcgccggttggcaaaccagttgtagacacgcacctccgtgacgaggttggagcccagcccctgtgcctgtgatggggacacccctctctggatgcattccgccctgcagaaatagccacccatgagcc

SEQ ID NO: 8 - DNA, 296 bp, Artificial Sequence
  target polynucleotide A8
ggctcatgggtggctatttctgcagggcggaatgcatccagagaggggtgtccccatcacaggcacaggggctgggctccaacctcgtcacggaggtgcgtgtctacaactggtttgccaaccggcgcaaagaagaagccttccggcacaagctggccatggacacgtacagcgggccccccccagggccaggcccgggacctgcgctgcccgctcacagctcccctggcctgcctccacctgccctctcccccagtaaggtccacggtaagtggtatgtggggacaagggacacg

SEQ ID NO: 9 - DNA, 202 bp, Artificial Sequence
  target polynucleotide A9
tacaagcaaggacactcaccagcttggcttctgtactcagcaggctgtggctgggctccaggcccgtgggggacacttggtggaggggtgtagacactgtcactaagggaccgccgctgcttgagggtacttctgcagtctcactggtcgcaggctgtccatagcgcacacctggagagggaagcagtgtcctgcctcagca

SEQ ID NO: 10 - DNA, 247 bp, Artificial Sequence
  target polynucleotide A10
atccccaccagcttaccgatgaccagggtggaggcacctgtgttggtgaacgtaggacccagggaggcaggctcaccaggcccgatggtcatgaccccaggaagtgaggccatgatgaggttctggggctgctggttgaggcctggggatgtctgctccaagctgtgcagtgctgtcagggtgctgacaggggggagggggcccccagctgctgagacctacgaggggaagccaagctgtgtccggg

SEQ ID NO: 11 - DNA, 247 bp, Artificial Sequence
  target polynucleotide A11
cccggacacagcttggcttcccctcgtaggtctcagcagctgggggccccctcccccctgtcagcaccctgacagcactgcacagcttggagcagacatccccaggcctcaaccagcagccccagaacctcatcatggcctcacttcctggggtcatgaccatcgggcctggtgagcctgcctccctgggtcctacgttcaccaacacaggtgcctccaccctggtcatcggtaagctggtggggat

SEQ ID NO: 12 - DNA, 191 bp, Artificial Sequence
  target polynucleotide A12
ggggctctgcagctgagccatggtggccatgaaggggctctgggtcacatggctctgcacaggtggcatgagcggctgctggtaggaggggtgcagcggctgggagaactggacgggctgcagggtggtcaggctgctgcccatgctgttgatgaccggcacactctgtgcctgcgtggaggccaggcctg

SEQ ID NO: 13 - DNA, 191 bp, Artificial Sequence
  target polynucleotide A13
caggcctggcctccacgcaggcacagagtgtgccggtcatcaacagcatgggcagcagcctgaccaccctgcagcccgtccagttctcccagccgctgcacccctcctaccagcagccgctcatgccacctgtgcagagccatgtgacccagagccccttcatggccaccatggctcagctgcagagcccc

SEQ ID NO: 14 - DNA, 378 bp, Artificial Sequence
  target polynucleotide A14
gggcagggacagtaagggagggggtggagccagggcctctcacctgtggggctggcgctgagccggtgggccggctgcaggtgctggatgctggcagggtcctggctggggacgtggagggtggtggcctgagatgccggcgtgtgaagcccggactcactggaggcctcagtgtctgaggtgaagacctgggggagcagagcagcctcctgagcccgggggatgtgggggtgaggggctctgtcacaggccaagggagggccagcaggcctggaccttacctgcttggtgggcgtgaggctggccagggcgctcaggttggtggtgtcggtgatgagcatagtctgcgggagcaggcccgtgtgggtgtactgggcc

SEQ ID NO: 15 - DNA, 378 bp, Artificial Sequence
  target polynucleotide A15
ggcccagtacacccacacgggcctgctcccgcagactatgctcatcaccgacaccaccaacctgagcgccctggccagcctcacgcccaccaagcaggtaaggtccaggcctgctggccctcccttggcctgtgacagagcccctcacccccacatcccccgggctcaggaggctgctctgctcccccaggtcttcacctcagacactgaggcctccagtgagtccgggcttcacacgccggcatctcaggccaccaccctccacgtccccagccaggaccctgccagcatccagcacctgcagccggcccaccggctcagcgccagccccacaggtgagaggccctggctccaccccctcccttactgtccctgccc

SEQ ID NO: 16 - DNA, 132 bp, Artificial Sequence
  target polynucleotide A16
gccttgtttgcctctgcagtgtcctccagcagcctggtgctgtaccagagctcagactccagcaatggccagagccacctgctgccatccaaccacagcgtcatcgagaccttcatctccacccagatggcc

SEQ ID NO: 17 - DNA, 539 bp, Artificial Sequence
  target polynucleotide A17
gcgtgggttgcgtttgcctgccggccggcagacacaaaccaaactccttgcacccactgcccccccaaaaccccactagccaagccctgtgggcacccccaacccccaaccctggccccagcggcccgtgaatcagggcccctgcctgttctgtttacattggagctggggaaattctccaaggttcatatttatccgtgtgcttagcgaagggactgaactttggacttcagccctgcaaagtgcaggcctcatggcagcggagagggacagggagctatggcctgcgatgggagtgggcagaggggagggtgggggaggcgcaggtgaggggccctgagcagctgggatggaaggtgctgggagccagggaggccatcaggagagcagcgttgggtcccacccccgaaagaaccttgtaggggcatcatgctgagacaggagagccaaaaatgcttgctgcttacgagagcggcagcagcagggagctctagacagctgggctagctcttctcaagggcagaggatgctcacggcca

SEQ ID NO: 18 - DNA, 539 bp, Artificial Sequence
  target polynucleotide A18
tggccgtgagcatcctctgcccttgagaagagctagcccagctgtctagagctccctgctgctgccgctctcgtaagcagcaagcatttttggctctcctgtctcagcatgatgcccctacaaggttctttcgggggtgggacccaacgctgctctcctgatggcctccctggctcccagcaccttccatcccagctgctcagggcccctcacctgcgcctcccccaccctcccctctgcccactcccatcgcaggccatagctccctgtccctctccgctgccatgaggcctgcactttgcagggctgaagtccaaagttcagtcccttcgctaagcacacggataaatatgaaccttggagaatttccccagctccaatgtaaacagaacaggcaggggccctgattcacgggccgctggggccagggttgggggttgggggtgcccacagggcttggctagtggggttttgggggggcagtgggtgcaaggagtttggtttgtgtctgccggccggcaggcaaacgcaacccacgc

SEQ ID NO: 19 - DNA, 12 bp, Artificial Sequence
  probe polynucleotide
agctcctggcgg

SEQ ID NO: 20 - DNA, 13 bp, Artificial Sequence
  probe polynucleotide
gcactgggtgagc

SEQ ID NO: 21 - DNA, 11 bp, Artificial Sequence
  probe polynucleotide
gggagtcctgc

SEQ ID NO: 22 - DNA, 16 bp, Artificial Sequence
  probe polynucleotide
gagcaaagaggcactg

SEQ ID NO: 23 - DNA, 12 bp, Artificial Sequence
  probe polynucleotide
aaggccccctgg

SEQ ID NO: 24 - DNA, 14 bp, Artificial Sequence
  probe polynucleotide
cagctggctcagtt

SEQ ID NO: 25 - DNA, 15 bp, Artificial Sequence
  probe polynucleotide
gcctggatcagtgcc

SEQ ID NO: 26 - DNA, 14 bp, Artificial Sequence
  probe polynucleotide
tggtgggccgcctc

SEQ ID NO: 27 - DNA, 15 bp, Artificial Sequence
  probe polynucleotide
cagaagggtctccac

SEQ ID NO: 28 - DNA, 12 bp, Artificial Sequence
  probe polynucleotide
ctcccccttgtc

SEQ ID NO: 29 - DNA, 15 bp, Artificial Sequence
  probe polynucleotide
agaagggtctccacc

SEQ ID NO: 30 - DNA, 16 bp, Artificial Sequence
  probe polynucleotide
atggtcaagtcctacc

SEQ ID NO: 31 - DNA, 15 bp, Artificial Sequence
  probe polynucleotide
ggaggtggtcgatac

SEQ ID NO: 32 - DNA, 13 bp, Artificial Sequence
  probe polynucleotide
accagtcccacct

SEQ ID NO: 33 - DNA, 12 bp, Artificial Sequence
  probe polynucleotide
gcagaagcgggc

SEQ ID NO: 34 - DNA, 11 bp, Artificial Sequence
  probe polynucleotide
gaagcgggccg

SEQ ID NO: 35 - DNA, 14 bp, Artificial Sequence
  probe polynucleotide
cagaagcgggccgc

SEQ ID NO: 36 - DNA, 12 bp, Artificial Sequence
  probe polynucleotide
cgggccgccctg

SEQ ID NO: 37 - DNA, 15 bp, Artificial Sequence
  probe polynucleotide
cagcgggaggtggtc

SEQ ID NO: 38 - DNA, 16 bp, Artificial Sequence
  probe polynucleotide
tggtcgataccactgg

SEQ ID NO: 39 - DNA, 14 bp, Artificial Sequence
  probe polynucleotide
acaagggcactccc

SEQ ID NO: 40 - DNA, 12 bp, Artificial Sequence
  probe polynucleotide
ccaacacctcaa

SEQ ID NO: 41 - DNA, 13 bp, Artificial Sequence
  probe polynucleotide
cgctgtgggatgt

SEQ ID NO: 42 - DNA, 13 bp, Artificial Sequence
  probe polynucleotide
cctcccgctgtgg

SEQ ID NO: 43 - DNA, 12 bp, Artificial Sequence
  probe polynucleotide
cacctcccgctg

SEQ ID NO: 44 - DNA, 14 bp, Artificial Sequence
  probe polynucleotide
aggtgggactggtt

SEQ ID NO: 45 - DNA, 13 bp, Artificial Sequence
  probe polynucleotide
acttacgctgcgc

SEQ ID NO: 46 - DNA, 12 bp, Artificial Sequence
  probe polynucleotide
cttcgccacacg

SEQ ID NO: 47 - DNA, 13 bp, Artificial Sequence
  probe polynucleotide
gtgttgggacagg

SEQ ID NO: 48 - DNA, 15 bp, Artificial Sequence
  probe polynucleotide
cggaggaaccgtttc

SEQ ID NO: 49 - DNA, 14 bp, Artificial Sequence
  probe polynucleotide
aggaggagcgagag

SEQ ID NO: 50 - DNA, 13 bp, Artificial Sequence
  probe polynucleotide
agcgagagacgct

SEQ ID NO: 51 - DNA, 13 bp, Artificial Sequence
  probe polynucleotide
agcgagagacgct

SEQ ID NO: 52 - DNA, 12 bp, Artificial Sequence
  probe polynucleotide
cacctgtgggct

SEQ ID NO: 53 - DNA, 12 bp, Artificial Sequence
  probe polynucleotide
ccgccccttctt

SEQ ID NO: 54 - DNA, 14 bp, Artificial Sequence
  probe polynucleotide
aaacggttcctccg

SEQ ID NO: 55 - DNA, 15 bp, Artificial Sequence
  probe polynucleotide
ccccacttgaaacgg

SEQ ID NO: 56 - DNA, 15 bp, Artificial Sequence
  probe polynucleotide
ctgcctctcataggc

SEQ ID NO: 57 - DNA, 13 bp, Artificial Sequence
  probe polynucleotide
atgctgggcccca

SEQ ID NO: 58 - DNA, 15 bp, Artificial Sequence
  probe polynucleotide
ctcctccactagcgt

SEQ ID NO: 59 - DNA, 15 bp, Artificial Sequence
  probe polynucleotide
gcggaatgcatccag

SEQ ID NO: 60 - DNA, 14 bp, Artificial Sequence
  probe polynucleotide
acctcgtcacggag

SEQ ID NO: 61 - DNA, 15 bp, Artificial Sequence
  probe polynucleotide
gaggtgcgtgtctac

SEQ ID NO: 62 - DNA, 16 bp, Artificial Sequence
  probe polynucleotide
caaccggcgcaaagaa

SEQ ID NO: 63 - DNA, 12 bp, Artificial Sequence
  probe polynucleotide
cccccagggcca

SEQ ID NO: 64 - DNA, 16 bp, Artificial Sequence
  probe polynucleotide
caactggtttgccaac

SEQ ID NO: 65 - DNA, 15 bp, Artificial Sequence
  probe polynucleotide
accggcgcaaagaag

SEQ ID NO: 66 - DNA, 15 bp, Artificial Sequence
  probe polynucleotide
cccccagtaaggtcc

SEQ ID NO: 67 - DNA, 12 bp, Artificial Sequence
  probe polynucleotide
gggcggaatgca

SEQ ID NO: 68 - DNA, 14 bp, Artificial Sequence
  probe polynucleotide
ctggatgcattccg

SEQ ID NO: 69 - DNA, 11 bp, Artificial Sequence
  probe polynucleotide
gcccagcccct

SEQ ID NO: 70 - DNA, 12 bp, Artificial Sequence
  probe polynucleotide
ctccgtgacgag

SEQ ID NO: 71 - DNA, 13 bp, Artificial Sequence
  probe polynucleotide
cacgcacctccgt

SEQ ID NO: 72 - DNA, 12 bp, Artificial Sequence
  probe polynucleotide
cacgcacctccg

SEQ ID NO: 73 - DNA, 15 bp, Artificial Sequence
  probe polynucleotide
tggcaaaccagttgt

SEQ ID NO: 74 - DNA, 12 bp, Artificial Sequence
  probe polynucleotide
ccccacgggcct

SEQ ID NO: 75 - DNA, 14 bp, Artificial Sequence
  probe polynucleotide
tcccccctgtcagc

SEQ ID NO: 76 - DNA, 14 bp, Artificial Sequence
  probe polynucleotide
cagacatccccagg

SEQ ID NO: 77 - DNA, 15 bp, Artificial Sequence
  probe polynucleotide
agcagccccagaacc

SEQ ID NO: 78 - DNA, 14 bp, Artificial Sequence
  probe polynucleotide
accagggtggaggc

SEQ ID NO: 79 - DNA, 14 bp, Artificial Sequence
  probe polynucleotide
cgcaggcacagagt

SEQ ID NO: 80 - DNA, 12 bp, Artificial Sequence
  probe polynucleotide
acccagaacccc

SEQ ID NO: 81 - DNA, 16 bp, Artificial Sequence
  probe polynucleotide
atgtgaccccgaaccc

SEQ ID NO: 82 - DNA, 11 bp, Artificial Sequence
  probe polynucleotide
gaccggcacac

SEQ ID NO: 83 - DNA, 12 bp, Artificial Sequence
  probe polynucleotide
gagcggctgctg

SEQ ID NO: 84 - DNA, 13 bp, Artificial Sequence
  probe polynucleotide
acgcccaccaagc

SEQ ID NO: 85 - DNA, 14 bp, Artificial Sequence
  probe polynucleotide
agacactgaggcct

SEQ ID NO: 86 - DNA, 13 bp, Artificial Sequence
  probe polynucleotide
cggcatctcaggc

SEQ ID NO: 87 - DNA, 12 bp, Artificial Sequence
  probe polynucleotide
ctgccggcatcc

SEQ ID NO: 88 - DNA, 13 bp, Artificial Sequence
  probe polynucleotide
ccggcccaccggc

SEQ ID NO: 89 - DNA, 13 bp, Artificial Sequence
  probe polynucleotide
cccaccggctcag

SEQ ID NO: 90 - DNA, 12 bp, Artificial Sequence
  probe polynucleotide
ccggctcagcgc

SEQ ID NO: 91 - DNA, 15 bp, Artificial Sequence
  probe polynucleotide
ccccacaggtgagag

SEQ ID NO: 92 - DNA, 13 bp, Artificial Sequence
  probe polynucleotide
tgggcgtgaggct

SEQ ID NO: 93 - DNA, 13 bp, Artificial Sequence
  probe polynucleotide
tctcgatgacgct

SEQ ID NO: 94 - DNA, 14 bp, Artificial Sequence
  probe polynucleotide
gagatgaaggtctc

SEQ ID NO: 95 - DNA, 13 bp, Artificial Sequence
  probe polynucleotide
ttgggggggcagt

SEQ ID NO: 96 - DNA, 18 bp, Artificial Sequence
  probe polynucleotide
tctgtttacattggagct